(19) United States
(12) Patent Application Publication
(10) Pub. No.: US 2012/0185170 A1
(43) Pub. Date: Jul. 19, 2012

(54) Profile Apparatus for In Situ Measurement of Sediment Oxygen Demand and Method of Using the Same

(76) Inventors: Robert J. Miskewitz, Cranford, NJ (US); Kelly L. Francisco, Hillsborough, NJ (US); Christopher G. Uchrin, Lebanon, NJ (US)

(21) Appl. No.: 13/298,456

(22) Filed: Nov. 17, 2011

Related U.S. Application Data
(60) Provisional application No. 61/415,088, filed on Nov. 18, 2010.

(51) Int. Cl. G01N 33/24; G06F 19/00

(52) U.S. Cl. 702/2; 73/61.65

(57) ABSTRACT

A profile apparatus that is useful in measuring sediment oxygen demand and methods utilizing the same.

### CROSS-REFERENCE TO RELATED APPLICATIONS

The present application claims the benefit of U.S. Provisional Patent Application No. 61/415,088, filed Nov. 18, 2010, the entire disclosure of which is incorporated by reference herein.

### GOVERNMENT RIGHTS STATEMENT

Pursuant to 35 U.S.C. §202(c) it is acknowledged that the U.S. Government has rights in the invention described, which was made in part with funds from the U.S. Geological Survey, Grant Number 2009NJ193B.

### FIELD OF INVENTION

The present invention is generally related to monitoring of sediment oxygen demand in aquatic systems. More specifically, the present invention provides an apparatus and method for determining the impact of flow rate upon sediment oxygen demand based upon dissolved oxygen transport through the sediment boundary layer in aquatic systems.

### BACKGROUND OF THE INVENTION

Sediment oxygen demand (SOD) is the sum of the dissolved oxygen removed from a water column by the respiration of organisms living in the sediment and the oxidation of reduced chemicals found in the sediment.^{[1]} The SOD in a stream can vary based on sediment age, depth of deposits, temperature, and chemical and biological characteristics. SOD is often considered an indicator of the health of an aquatic system because sediment populations remain relatively stable while the overlying water can be transient.^{[2]} Therefore SOD measurement is an important part of any water quality investigation of aquatic systems, especially riverine systems, and is an important parameter that must be integrated into numerical simulations of water quality. Virtually all models attempt to observe mass-balance principles within the water column, but often fail to close mass balances involving interaction with the sediment.^{[3]} Several water quality models utilize the sediment digenesis model presented by DiToro and Fitzpatrick to simulate the processes that contribute to SOD.^{[4]} Park et al. simulated SOD in a large estuarine system by using the sediment digenesis model incorporated into a Three-Dimensional Hydrodynamic-Eutrophication Model (HEM-3D).^{[5]} To ensure that SOD values based on predictions using numerical hydrodynamic models are representative of field conditions, accurate measurements of SOD must be collected.

There are no currently accepted standard methods for field measurements of in-situ SOD.^{[6]} The most common method uses a sediment chamber of a known volume which is lowered from the water surface to the sediment. Enclosed in the chamber are a dissolved oxygen probe and a mixing device. The system is left in place for a period of approximately 2 hours during which the dissolved oxygen in the chamber is periodically measured.^{[7,8]} Dissolved oxygen is removed from the water column and consumed in the benthic layer. The rate of oxygen depletion in the water is used to calculate the SOD. Chamber method measurements have been used in previous studies of SOD.^{[1, 2, 9-13]}

The most important environmental factor that will affect the SOD is the flow rate of water over the sediment/water interface.^{[14]} The flow conditions also represent one of the largest sources of uncertainty in SOD measurements. It has been observed that SOD increases proportionally to stream velocity^{[7]} especially at low velocities.^{[15]} This dependence on flow is thought to be related to mixing in the near surface boundary layer, however it has also been shown that even small changes in the stream velocity impact the benthic respiration as well as the chemical oxygen demand in sediments.^{[16]} Studies have attempted to reproduce natural flow conditions in a chamber by creating an equivalent shear but were not particularly successful because the methodology required a great deal of information about the flow conditions and was imprecise since the chamber itself could not sustain the streamlines present in a natural system.^{[17,18]}

The development of an improved apparatus and method for measuring sediment oxygen demand, and thereby the health of an aquatic system, where time or terrain is prohibitive to previous methodologies, is highly desired. Such an improved invention would enable measurements of SOD in less than 15 minutes, provide measurements under almost any condition present in an aquatic system, and could be used to determine the relationship between flow rate and SOD at a particular site.

### SUMMARY OF THE INVENTION

The present invention comprises an apparatus and method for the in situ measurement of SOD in an aquatic system. The SOD measurement of the present invention is based upon a characterization of the flow in the near sediment boundary layer and the transport of dissolved oxygen down a concentration gradient. This invention can be useful in determining the health and status of an aquatic system by determining the SOD. The profile apparatus of the present invention for use in measuring sediment oxygen demand in an aquatic system comprises:

(a) a support for positioning the profile apparatus in the aquatic system;

(b) a dissolved oxygen measurement system connected to the support for measuring a dissolved oxygen gradient; and

(c) a water flow velocity measurement system connected to the support and proximate to the dissolved oxygen measurement system for measuring water flow velocities in at least two directions. In one embodiment, the water flow velocities in at least two directions are orthogonal. In another embodiment the profile apparatus of the invention further comprises a computer processor in communication with the dissolved oxygen measurement system and the water flow velocity measurement system, for receiving and processing data produced by the dissolved oxygen measurement system and the water flow velocity measurement system, to calculate the sediment oxygen demand of the aquatic system.

The present invention provides a method for measuring sediment oxygen demand in an aquatic system comprises the steps of:

(a) positioning the profile apparatus of the present invention in the aquatic system;

(b) measuring a dissolved oxygen concentration gradient for a pre-determined time period;

(c) measuring water flow velocities in at least two directions for the time period; and

(d) determining the sediment oxygen demand from the dissolved oxygen concentration gradient, and the water flow velocities measured over the time period.

Use of the present invention takes less than fifteen minutes and provides SOD measurements under almost any condition present in a stream or a particular site. The main advantage that this invention has over chamber methods of the prior art is the ability to measure the SOD flux as a function of the flow.

### DESCRIPTION OF DRAWINGS AND TABLES

FIG. 1. A schematic of the profile sediment oxygen demand (SOD) apparatus.

FIG. 2. A graph displaying a representative example of dissolved oxygen profile, during a morning deployment, as a function of time.

FIG. 3. A graph demonstrating a comparison between sediment oxygen demand (SOD) and friction velocity, u*.

### DETAILED DESCRIPTION OF THE INVENTION

SOD is the sum of the dissolved oxygen removed from a water column by the respiration of organisms living in the sediment and the oxidation of reduced chemicals found in the sediment. SOD is often considered an indicator of the health of an aquatic system because sediment remains relatively stable while the overlying water can be transient. Therefore SOD measurement is an important part of any water quality investigation of aquatic systems and is an important parameter that must be integrated into numerical simulations of water quality.

As used herein, the term “aquatic system,” refers to a body of water that may be composed of predominantly freshwater or saltwater. Such a body of water may be a lotic system (i.e. running or flowing water). Such a lotic system may be temporary or permanent. Examples of lotic systems may include rivers, streams, springs, creeks, estuaries and the like. Preferably, the aquatic systems of the present invention are rivers or streams. As used herein, the term “aquatic system bed,” refers to the bottom of the aquatic system.

The present invention provides an apparatus and method for the in situ measurement of SOD in an aquatic system. The profile methodology is based upon transport through the logarithmic boundary layer that develops when fluid flows over a flat plate. The method takes advantage of the fact that the flux, q_{SOD}, and the gradient of dissolved oxygen, dC/dz are proportional in the boundary layer. The constant of proportionality is a diffusion coefficient. If the stream flow is laminar, the coefficient is the molecular diffusion coefficient for oxygen in water, but in order to properly represent a realistic stream flow, a turbulent diffusion coefficient must be used. In this case, the turbulent diffusion coefficient is referred to as the vertical eddy diffusivity, ε_{z}.

$${q}_{SOD} = {ɛ}_{z} \frac{d C}{d z} \qquad ( 2 )$$

The vertical eddy diffusivity is not a function of dissolved oxygen transport parameters, but rather the turbulence present in the flow itself.^{[19]} An equation to calculate the vertical eddy diffusivity calculates the vertical eddy diffusivity as a function of the friction velocity, u*, elevation above the bed, z, and the depth, d, of the water.

$${ɛ}_{z} = κ {u}^{*} z ( 1 - \frac{z}{d} ) \qquad ( 3 )$$

κ is the von Karmen constant which has a value of 0.4.

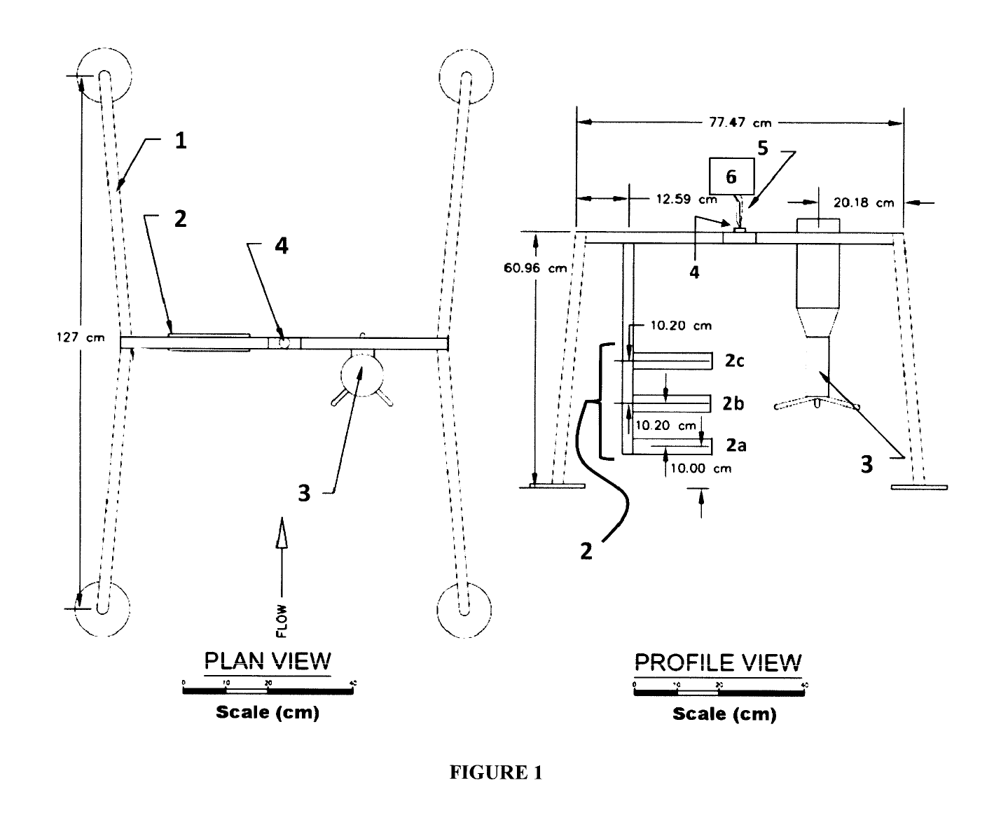

FIG. 1 is a schematic of a representative profile SOD apparatus of this invention. Referring to FIG. 1, the profile apparatus comprises a support 1 for positioning the profile apparatus in an aquatic system, a dissolved oxygen measurement system 2 connected to the support 1 for measuring dissolved oxygen, and a water flow velocity measurement system 3 connected to the support 1 and proximate to the dissolved oxygen measurement system 2 for measuring the water flow velocities in at least two directions. The profile SOD apparatus may also include a computer processor 6 in communication with the dissolved oxygen measurement system 2 and the water flow velocity measurement system 3 for receiving and processing the data from the systems 2 and 3 in order to calculate the sediment oxygen demand of the aquatic system. Specifically, the computer processor 6 may be attached directly to the support 1, wherein it is exposed to the aquatic system and may communicate with both the dissolved oxygen measurement system 2 and the water flow velocity measurement system 3, to collect data, which is stored electronically, and retrieved upon removal of the profile SOD apparatus from a particular site. In a preferred embodiment, the profile SOD apparatus comprises an opening for port connections 4 on the support 1 which allows for a tether and cables 5 to exit the support 1 and connect to a computer processor 6 which may be positioned remotely, outside of the aquatic system, to allow for communication between the computer processor 6 and both the dissolved oxygen measurement system 2 and the water flow velocity measurement system 3. The computer processer 6 which is in communication with the dissolved oxygen measurement system 2 and the water flow velocity measurement system 3 may communicate with systems 2 and 3 by any means known in the art including communication by wire or cable, electrooptical communication, wireless communication or a combination thereof.

The dissolved oxygen measurement system 2 and the water flow velocity measurement system 3 used in the apparatus may be connected to the support 1 which may be constructed of black iron 1″ pipe with galvanized iron fittings to resemble a sawhorse. The legs of the support 1, as a sawhorse, can be located sufficiently distant from the sensors to avoid any disturbance to the sediment of the aquatic system bed and the flow of water. However, in constructing a support, any structure, scaffold or lattice which supports systems 2 and 3, disclosed herein, and positions the profile apparatus in the aquatic system would be suitable.

The gradient of dissolved oxygen concentrations may be measured via a dissolved oxygen measurement system 2 which is composed of at least two dissolved oxygen probes 2a,2b. Preferably the dissolved oxygen measurement system will be composed of three dissolved oxygen probes 2a, 2b, 2c. Each dissolved oxygen probe 2a,2b,2c measures the concentration of dissolved oxygen in the surrounding water. Defining the relative positions of the dissolved oxygen probes, when there are at least two dissolved oxygen probes 2a,2b, the first dissolved oxygen probe 2a may be positioned in closer proximity to the aquatic system bed than the second dissolved oxygen probe 2b. Additionally, the at least two dissolved oxygen probes may be connected to the support such that they are aligned with respect to each other wherein the first dissolved oxygen probe 2a is positioned directly below the second dissolved oxygen probe 2b. When there are at least three dissolved oxygen probes, the third dissolved oxygen probe 2c may be positioned such that it is further removed in proximity from the aquatic system bed than the second dissolved oxygen probe 2b. In an embodiment wherein the dissolved oxygen measurement system 2 is comprised of at least three dissolved oxygen probes, the third dissolved oxygen probe 2c may be connected to the support such that it is aligned with either the first dissolved oxygen probe 2a and/or the second dissolved oxygen probe 2b which are both beneath the third dissolved oxygen probe 2c. The dissolved oxygen probes of the dissolved oxygen measurement system 2 should be placed within at least about 35 cm of the aquatic system bed. For example, when using two dissolved oxygen probes, a first probe may be placed at 10 cm above the aquatic system bed and a second probe may be placed approximately 25 cm above the first probe, and approximately 35 cm above the aquatic system bed. In a particularly preferred embodiment, the dissolved oxygen probes of dissolved oxygen measurement system 2 may be three RDOG Pro optical dissolved oxygen probes (In Situ, Inc.) installed at spaced apart positions on a rack at 10 cm, 20.8 cm and 31.6 cm, respectively, above the aquatic system bed (i.e., sediment surface). The dissolved oxygen gradient is measured at a rate of 1 Hz and averaged over a one minute period. The SOD flux is then calculated for the length of the deployment.

In order to calculate the vertical eddy diffusivity, the friction velocity, u*, must be measured.^{[20]} The friction velocity is determined by taking the square root of the covariance of the turbulent fluctuations in the vertical and horizontal water flow velocities,

$${u}^{*} = \sqrt{\overline{{u}^{′} {w}^{′}}} . \qquad [ 21 ]$$

The water flow velocities are collected using a water flow velocity measurement system 3 which measures the water flow velocity in at least two directions. Preferably, the water flow velocities in at least two directions are orthogonal. In one embodiment, the water flow velocity measurement system 3 measures the water flow velocity in at least three directions, wherein the three directions are orthogonal with respect to each other. In another embodiment, the water flow velocity measurement system 3 comprises a first velocimeter for measuring a water flow velocity in a first direction, a second velocimeter for measuring a water flow velocity in a second direction, wherein the first and second water flow velocity directions are orthogonal with respect to each other and, optionally, a third velocimeter for measuring a water flow velocity in a third direction which is orthogonal to the first and second water flow velocity directions.

In a preferred embodiment, the water flow velocities in two or more directions are measured by a single velocimeter which is proximate to the dissolved oxygen measurement system 2 and may be proximate to or, preferably, connected to the support 1. In a particularly preferred embodiment, the water flow velocity measurement system 3 is a Sontek® Acoustic Doppler Velocimeter (ADV®) (Xylem Inc.) which measures water flow velocity in at least two orthogonal directions at a point at least about 15 cm above the aquatic system bed (i.e. sediment surface) at a resolution of 10 Hz. The dissolved oxygen probes of the dissolved oxygen measurement system 2 can be oriented perpendicular to the water current and located at least about 5 cm away from the sample volume of the ADV. The dissolved oxygen probes should be close enough to be sampling the same water as the ADV, but far enough away to avoid turbulent impacts caused by the sensor bodies.

ADV measurements are performed by transmitting an ultrasonic signal into the water and measuring the Doppler shift of the signal as it reflects back to the sensor from particles present in the water column.^{[22]} Using these measurements, 10 minute average u* values are calculated. The u* values are then used to calculate the eddy diffusivity. Since the dissolved oxygen gradient measurements may be calculated using three probes 10.8 cm apart, for example, the average eddy diffusivity is calculated by integrating equation (2) between the probes and dividing by the interval. In this way the average flux is calculated between the probes.

$${q}_{SOD} = {ɛ}_{z} \frac{Δ C}{Δ z} \qquad ( 4 )$$

In one embodiment, the profile SOD method disclosed herein may be performed at a given location for a pre-determined time period of at least about one minute to several days. Preferably, the pre-determined time period is at least about 5 to 15 minutes. In another embodiment, the profile SOD apparatus may be installed at a fixed location and the SOD may be monitored continuously.

The details of this method including results were published in: Miskewitz, R., Francisco, K. and Uchrin, C. Comparison of a novel profile method to standard chamber methods for measurement of Sediment Oxygen Demand, Journal of Environmental Science and Health, Part A. 45:795-802 (2010).

The following example is provided to describe the invention in further detail. This example is provided for illustrative purposes only and is not intended to limit the invention in any way.

### Example

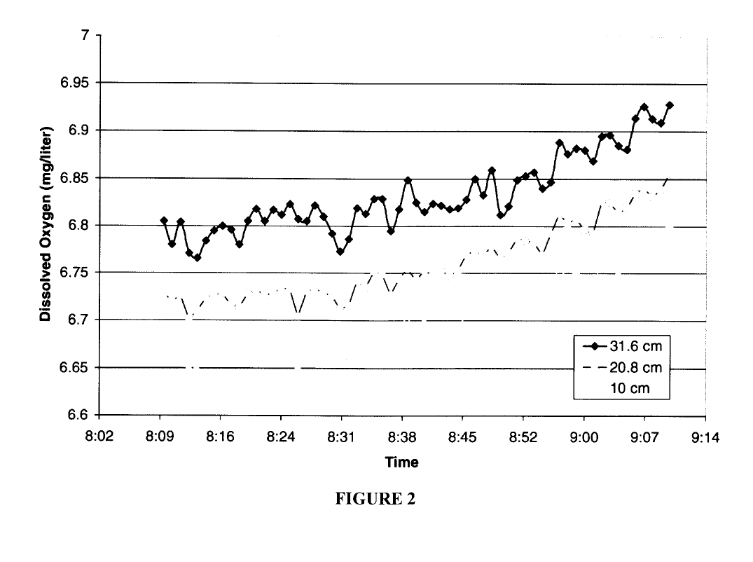

In a first trial, SOD was measured using both the profile and chamber method over three days at two locations. In a second trial, SOD was measured by only the profile method in an urban creek over five days at four locations. Deployments were conducted for a period of approximately 6 hours each. During this time at least two chamber measurements were attempted and the profile was sampled continuously. The data used to calculate each SOD flux are included in Table 1. Table 1 also shows the measurements of the friction velocity and average dissolved oxygen concentration at the topmost probe, which are closest to the overall water column concentrations. Chamber and profile systems were installed two meters apart, laterally across the stream, from each other. Measurements were collected generally between 9:30 to 14:00 on each day. Measurements collected while personnel were in the stream to move the chamber were discarded. A total of seven chamber SOD measurements were collected over three days, including one in the Millstone River and six in the Lawrence Brook. Meanwhile a total of 50 profile SOD measurements were collected, including eight in the Millstone River. During each measurement interval, 1-minute averaged dissolved oxygen concentrations were collected at each probe. The gradient between probes remained relatively constant despite changing dissolved oxygen concentrations in the stream (FIG. 2).

In the first trial, chamber measurements in the Lawrence Brook varied from 2.7 to 8.9 g/m²/day and had a mean of 5.0 g/m²/day. Concurrent profile measurements varied from 1.3 to 13.5 g/m²/day and had a mean of 7.16 g/m²/day. In the Millstone River, the chamber measurement was 4.6 g/m²/day while the profile measurements varied from 0.5 to 2.2 g/m²/day with a mean of 1.32 g/m²/day. The measurements made via both profile and chamber methods were consistent with previous, published values for similar streams (Table 2). In the second trial, profile measurements at sites A-D in the urban creek displayed mean SOD fluxes which varied from 2.5 to 12.22 g/m²/day with an average mean SOD flux among sites A-D of 7.14 g/m²/day for the urban creek (Table 3).

TABLE 1
Data Collected on Lawrence Brook and Millstone River (First Trial).

| | | | | |
|---|---|---|---|---|
| | | DO | | Eddy |
| Time | Friction velocity | concentration | DO gradient | diffusivity |
| of Day | (cm/s) | (mg/L) | (mg/L/cm) | (cm²/s) |

Millstone River

| | | | | |
|---|---|---|---|---|
| 9:15 | 0.233 | 5.06 | 0.00180 | 1.31 |
| 9:25 | 0.217 | 5.05 | 0.00144 | 1.22 |
| 9:35 | 0.226 | 5.04 | 0.00188 | 1.27 |
| 9:45 | 0.210 | 5.05 | 0.00195 | 1.17 |
| 9:55 | N/A | 5.06 | 0.00192 | N/A |
| 10:05 | 0.112 | 5.08 | 0.00204 | 0.62 |
| 10:15 | 0.059 | 5.11 | 0.00210 | 0.33 |
| 10:25 | 0.250 | 5.13 | 0.00225 | 1.40 |
| 10:35 | 0.106 | 5.16 | 0.00223 | 0.59 |
| 10:45 | 0.133 | 5.18 | 0.00221 | 0.74 |

Lawrence Brook

| | | | | |
|---|---|---|---|---|
| 9:00 | 0.193 | 6.90 | 0.00621 | 1.19 |
| 9:10 | 0.338 | 6.84 | 0.00393 | 2.08 |
| 9:20 | 0.203 | 6.95 | 0.00544 | 1.25 |
| 9:30 | 0.241 | 6.99 | 0.00569 | 1.48 |
| 9:51 | 0.283 | 7.06 | 0.00539 | 1.74 |
| 10:01 | 0.337 | 7.10 | 0.00526 | 2.08 |
| 10:11 | 0.354 | 7.17 | 0.00564 | 2.18 |
| 10:21 | 0.460 | 7.20 | 0.00538 | 2.83 |
| 10:31 | 0.352 | 7.26 | 0.00554 | 2.17 |
| 10:41 | 0.283 | 7.29 | 0.00581 | 1.74 |
| 10:51 | 0.321 | 7.34 | 0.00566 | 1.98 |
| 11:01 | 0.416 | 7.37 | 0.00546 | 2.56 |
| 11:11 | 0.446 | 7.40 | 0.00553 | 2.75 |
| 11:21 | 0.458 | 7.44 | 0.00505 | 2.82 |
| 11:31 | 0.463 | 7.47 | 0.00545 | 2.85 |
| 9:26 | 1.636 | 7.33 | 0.00196 | 10.07 |
| 9:36 | 1.710 | 7.36 | 0.00222 | 10.53 |
| 9:46 | 1.711 | 7.38 | 0.00228 | 10.53 |
| 9:56 | 1.742 | 7.40 | 0.00233 | 10.73 |
| 10:06 | 1.726 | 7.42 | 0.00227 | 10.63 |
| 11:50 | 0.368 | 7.53 | 0.00575 | 2.26 |
| 12:00 | 0.322 | 7.56 | 0.00512 | 1.98 |
| 12:10 | N/A | 7.58 | 0.00469 | N/A |
| 12:20 | 0.147 | 7.59 | 0.00490 | 0.90 |
| 12:30 | 0.290 | 7.61 | 0.00571 | 1.79 |
| 12:40 | 0.072 | 7.61 | 0.00479 | 0.45 |
| 12:50 | N/A | 7.62 | 0.00427 | N/A |
| 13:00 | 0.253 | 7.65 | 0.00507 | 1.56 |
| 13:10 | 0.589 | 7.64 | 0.00395 | 3.63 |
| 13:20 | 0.335 | 7.66 | 0.00454 | 2.06 |
| 13:30 | 0.346 | 7.69 | 0.00578 | 2.13 |
| 13:40 | 0.356 | 7.68 | 0.00513 | 2.19 |
| 13:50 | 0.303 | 7.69 | 0.00466 | 1.86 |
| 14:00 | N/A | 7.69 | 0.00484 | N/A |
| 14:10 | 0.411 | 7.68 | 0.00452 | 2.53 |
| 14:20 | N/A | 7.69 | 0.00490 | N/A |
| 14:30 | 0.269 | 7.70 | 0.00542 | 1.66 |
| 12:01 | 0.703 | 7.48 | 0.00275 | 4.33 |
| 12:11 | 0.725 | 7.47 | 0.00283 | 4.46 |
| 12:21 | 0.649 | 7.47 | 0.00281 | 3.99 |
| 12:31 | 0.651 | 7.46 | 0.00281 | 4.01 |
| 12:41 | 0.632 | 7.45 | 0.00263 | 3.89 |
| 12:51 | 0.592 | 7.44 | 0.00271 | 3.65 |
| 13:01 | 0.659 | 7.43 | 0.00265 | 4.06 |
| 13:11 | 0.589 | 7.41 | 0.00266 | 3.62 |

Note:
DO Concentration listed is the measurement of the topmost probe during deployment.

TABLE 2
SOD measurements (First Trial).
SOD Flux measurements (g/m²/day)

| | | |
|---|---|---|
| Millstone River | Chamber Method | 4.6 |
| | Profile Method | 0.5-2.2 |
| Lawrence Brook | Chamber Method | 3.6-9.5 |
| | Profile Method | 1.3-10.1 |
| Lawrence Brook | Chamber Method | 6.7-13.0 |
| | Profile Method | 4.7-13.5 |
| Passaic River NJ (freshwater)^{[9]} | 1983 | 0-2.43 |
| Dead River, NJ^{[1]} | 2005 | 5.11-17.34 |
| Black Creek, SC^{[26]} | 1980 | 0.8-9.34 |

TABLE 3
Mean SOD Flux Measurements (Second Trial)

| | SOD Flux |
|---|---|
| Site | (g/m2/day) |
| A | 6.95 |
| B | 10.47 |
| C | 4.4 |
| D | 6.29 |
| A | 2.5 |
| D | 12.22 |

The streams are different in character and size. In the streams of the first trial, the Millstone River drains an area of 608 km² and its bed can be characterized as sandy. Installing the chamber was difficult due to the fact that the sandy bottom resulted in leaks. The chamber was pushed into the sediment to create a seal. Some disruption of the sediments may have occurred in the effort to secure a good seal along the bottom of the chamber and resulted in exposure of buried sediments which may have impacted the SOD measurements. The Lawrence Brook, by contrast, drains a much smaller area of 96 km² that is comprised of more suburban and urban areas. The bottom sediments in the Lawrence Brook are silty and contain a high amount of organic matter and it was much easier to obtain a good seal for the chamber. In the second trial, the stream was located in an urban environment. The stream located in the urban environment received runoff from a large urban area and is subject to intermittent combined sewer overflow discharges. The bottom sediments at the sampling locations varied from sandy bottom (Table 3; site A) to organic mud (Table 3; site D).

Although the SOD measurements made via the profile method are very close to those made with the chamber method, significant differences were noted. Those differences presumably occurred from the impacts of velocity. SOD increases proportionally to stream velocity.^{[7]} A well-developed flow regime that can simulate the existing stream conditions is practically impossible in a chamber. It is common practice to mix the water in the chamber at a high enough rate to approximate a completely mixed condition.^{[2]} In this condition, all resistance to the flux on the water side of the sediment/water interface is removed and the SOD is a completely sediment side controlled, zero order process. Thus, the measured SOD represents the maximum value that can be attained. Determining the velocity required to attain the completely mixed condition requires mixing up to a point that will disturb the sediment thereby changing the system. If however, a completely mixed condition is not present, chamber measurements may underestimate the maximum SOD due to stratification effects. The profile SOD method measures actual SOD as a function of stream flow and eliminates error associated with chamber mixing.

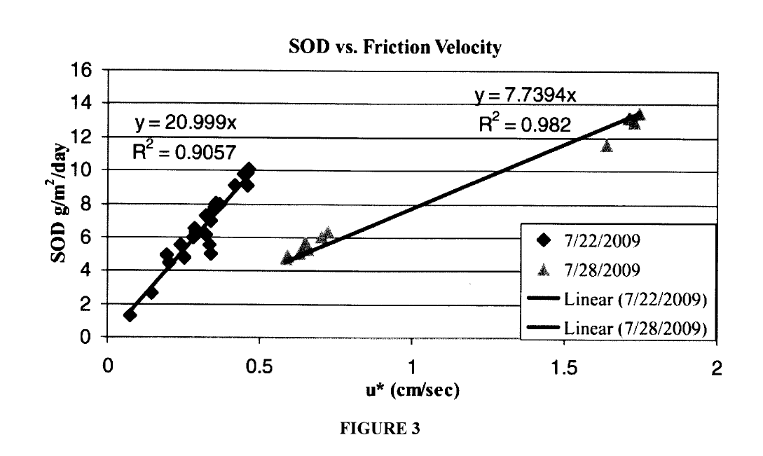

During the 2 days that the profile and chamber systems were deployed in the Lawrence Brook stream conditions varied greatly. There was a storm the previous day and the stream flow was elevated during the first half of the deployment (9 AM-11 AM); it returned to a more normal condition within a few hours (12 Noon). As a result, measurements collected on both of these days exhibited a fairly wide range of flow conditions. Flow conditions were compared to SOD using the friction velocity, u*. By definition, it is constant over the height of the boundary layer and therefore yields a more consistent parameter than the measured velocity and is a direct measure of mixing in the water column. A linear relationship of u* with SOD is clearly seen on both of the two days of measurement in the Lawrence Brook (FIG. 3).

Development of a model of SOD as a function of the friction velocity in stream would enable more accurate prediction of the dissolved oxygen in the system. Murphy and Hicks stated that for modeling purposes, simulation of bottom velocities as near as possible to field conditions should be the goal of any SOD experiment^{[8]} The invention enables not only measurement of SOD at field conditions but could also enable a SOD−u* rating curve to be developed.

It is also clear that the magnitude of SOD can be impacted by disturbance of the surface of the benthic layer.^{[1, 13]} The instances of disturbed sediments in this study resulted from increased suspension of sediment during elevated runoff events and from placement of the chamber for SOD measurement. During the week between the two sampling events on the Lawrence Brook, there was a rainfall of 4.39 centimeters and the flow in the stream was elevated. This storm event disturbed the sediment surface thus changing the magnitude of SOD with respect to the mixing in the boundary layer. After the storm event, the friction velocity required for SOD of a similar magnitude a week prior, was significantly higher. This result agrees with the premise that during the storm larger particles such as sand were suspended due to elevated flows and settled out as the flow rate decreased. These larger particles are commonly associated with lower SOD and thus greater velocities in the chamber are required for increased SOD.

Measurements made with the chamber were also apparently impacted by disturbance of the sediments, especially during chamber placement. One reason for this is that the topmost layer of undisturbed sediments is assumed to be mostly oxidized and results in a relatively smaller SOD on the overlying water column. Once disturbed, the organic-rich sediments that underlie the topmost sediments are exposed to the oxygen rich water and SOD increases. On one day, one of the fluxes measured in the chamber was higher than expected. This was presumably the result of poor chamber installation due to elevated flow and deep water. It is hypothesized that benthic sediments underlying the surface were exposed during placement.

This invention presents a methodology for measuring SOD that could incorporate the effects of stream velocity. Currently, the vast majority of SOD measurements are taken via the chamber method of Murphy and Hicks.^{[8]} SOD chamber measurements were collected alongside dissolved oxygen profile SOD measurements. The profile measurements were found to be in relative agreement with the chamber measurements, as well as documented measurements in previous studies. The advantage of the profile method is that it takes into account the dependence of SOD on flow. In addition, much higher temporal resolution can be attained with SOD measurements. SOD measurement can be obtained in 10 minutes rather than 2 hours when using the chamber method. Another important advantage of the new method is that there is less impact to the benthic layer when taking the measurement, thereby yielding a result that more closely approximates the natural SOD present in the stream.

The foregoing specification includes citations to certain literature references, which are provided to indicate the state of the art to which this invention pertains. The entire disclosure of each of the cited references is incorporated by reference herein. While various embodiments of the present invention have been described and/or exemplified above, numerous other embodiments will be apparent to those skilled in the art upon review of the foregoing disclosure. The present invention is, therefore, not limited to the particular embodiments described and/or exemplified, but is capable of considerable variations and modifications without departure from the scope of the appended claims.

Furthermore, the transitional phrases “comprising”, “consisting essentially of” and “consisting of” define the scope of the appended claims, in original and amended form, with respect to unrecited additional claim elements or steps. The term “comprising” is intended to be inclusive or open-ended and does not exclude additional unrecited elements, method steps or materials. The phrase “consisting of” exquisite any element, step one material other than those specified in the claim, and, in the latter instance, impurities ordinarily associated with specified materials. The phrase “consisting essentially of” limits the scope of a claim to the specified elements, steps or materials and those that do not materially affect the basic and novel characteristic(s) of the claimed invention. All apparatuses and processes identified herein can, in alternative embodiments, be more specifically defined by any of the transitional phrases “comprising”, “consisting essentially of” and “consisting of”.

### REFERENCES

1. Otubu, J. E., Hunter, J. V., Francisco, K. L. and Uchrin, C. G. (2006) Temperature Effects On Tubificid Worms And Their Relation To Sediment Oxygen Demand. J. Environ. Sci. Health Pt. A 41:8, pp. 1607-1613.
2. Uchrin, C. G. and Ahlert, W. K. (1985) In Situ Sediment Oxygen Demand Determinations In The Passaic River (NJ) During The Late Summer/Early Fall 1983. Water Res. 19:9, pp. 1141-1144.
3. Shanahan, P., Henze, M., Koncsos, L., Rauch, W., Reichert, P., Somlyody, L. and Vanrolleghem, P. Grabow, W. O. K., Dohmann, M. Haas, C. et al. (eds) (1998) River Water Quality Modelling: Ii. Problems Of The Art; pp. 245-252. Elsevier Science Ltd., Oxford, UK.
4. Di Toro, D. M. and Fitzpatrick, J. J. (1993) Chesapeake Bay Sediment Flux Model U.S. Army Engineer District, Baltimore, Md—A981762. Prepared For The U.S. Environmental Protection Agency And U.S. Army Engineer District: Baltimore, Md., 1993.
5. Park, S., Najjar, K. and Uchrin, C. (1995) A Water Quality Management Model For The Lakes Bay Estuarine Embayment 2: Hydrodynamic Tidal Model. J. Environ. Sci. Health Pt. A A30:5, pp. 1025-1038.
6. Ziadat, A. H. and Berdanier, B. W. (2004) Stream Depth Significance During In-Situ Sediment Oxygen Demand Measurements In Shallow Streams. J Am Water Resour Assoc. 40:1, pp. 631-638.
7. Truax, D. D., Shindala, A., Sartain, H. and Truax, D. D. (1995) Comparison Of Two Sediment Oxygen Demand Measurement Techniques. J. Environ. Eng. 121:9, p. 619.
8. Murphy, P j and Hicks, D b. Hatcher, K. J. (ed) (1986) In-Situ Method For Measuring Sediment Oxygen Demand. Sediment Oxygen Demand: Processes, Modeling And Measurement; University Of Georgia Institute Of Natural Resources, Athens, Ga.
9. Giga, J. V. and Uchrin, C. G. (1990) Laboratory And In Situ Sediment Oxygen Demand Determinations For A Passaic River (Nj) Case Study. J. Environ. Sci. Health Pt. A 25:7, p. 845.
10. Uchrin, C. G., Hunter, M. J. G., Park, S. S. and Vadas, T. M. (2005) In Situ Measurement Of Macrophyte Photosynthesis And Respiration In Shallow Lakes. J. Environ. Eng. 131:2, pp. 315-319.
11. Rounds, S. A. and Doyle, M. C. (1997) Sediment Oxygen Demand In The Tualatin River Basin, Oregon, 1992-96 U.S. Geological Survey, Reston, Va.—Wri 97-4103
12. Caldwell, J. M. and Doyle, M. C. (1995) Sediment Oxygen Demand In The Lower Willamette River, Oregon, 1994 U.S. Geological Survey, Reston, Va.—Wri 95-4196.
13. Wood, T. M. (2001) Sediment Oxygen Demand In Upper Klamath And Agency Lakes, Oregon, 1999 U.S. Geological Survey, Reston, Va.—Wri 2001-4080.
14. Josiam, R. M. and Stefan, H. G. (1999) Effect Of Flow Velocity On Sediment Oxygen Demand: Comparison Of Theory And Experiments. J. Am. Water Resour Assoc. 35:2, pp. 433-440.
15. Higashino, M. (2004) Unsteady Diffusional Mass Transfer At The Sediment/Water Interface: Theory And Significance For Sod Measurement. Water Res. 38:2, pp. 1-12.
16. Boynton, W. R., Kemp, W. M., Osborne, C. G., Kaumeyer, K. R. and Jenkins, M. C. (1981) Influence Of Water Circulation Rate On In Situ Measurements Of Benthic Community Respiration. Mar. Biol. 65:2, pp. 185-190.
17. Jubb, S. (2001) Relating Oxygen Demand To Flow: Development Of An In Situ Sediment Oxygen Demand Measurement Device. Water Sci. Technol. 43:5, pp. 203-210.
18. Arega, F. and Lee, J. H. W. (2005) Diffusional Mass Transfer At Sediment-Water Interface Of Cylindrical Sediment Oxygen Demand Chamber. J. Environ. Eng. 131:5, pp. 755-766.
19. Fischer, H. B. (1979) Mixing In Inland And Coastal Waters; p. 483. Academic Press, New York.
20. Elder, J. W. (1959) The Dispersion Of Marked Fluid In Turbulent Shear Flow. J. Fluid Mech. 5:3, pp. 544-560.
21. Schlichting, H. (1979) Boundary-Layer Theory. Mcgraw-Hill Series In Mechanical Engineering; p. 817. Mcgraw-Hill, New York.
22. Voulgaris, G. and Trowbridge, J. H. (1998) Evaluation Of The Acoustic Doppler Velocimeter (Adv) For Turbulence Measurements. J. Atmosph. Oceanic Tech. 15, pp. 272-289.

We claim:

1. A profile apparatus for use in measuring sediment oxygen demand in an aquatic system, comprising:
(a) a support for positioning the profile apparatus in the aquatic system;
(b) a dissolved oxygen measurement system connected to the support for measuring a dissolved oxygen concentration gradient; and
(c) a water flow velocity measurement system connected to the support and disposed in proximity to the dissolved oxygen measurement system for measuring water flow velocities in at least two directions.

2. The profile apparatus of claim 1, wherein the at least two directions are orthogonal.

3. The profile apparatus of claim 1, the dissolved oxygen measurement system comprising at least a first dissolved oxygen probe and a second dissolved oxygen probe.

4. The profile apparatus of claim 3, wherein the first probe is positioned in closer proximity to the aquatic system bed than the second probe.

5. The profile apparatus of claim 4, wherein the at least two probes are connected to the support such that they are aligned with respect to each other.

6. The profile apparatus of claim 3, wherein the first probe and the second probe are positioned such that they are within at least about 35 cm of the aquatic system bed.

7. The profile apparatus of claim 1, wherein the dissolved oxygen measurement system comprises at least a first dissolved oxygen probe, a second dissolved oxygen probe and a third dissolved oxygen probe.

8. The profile apparatus of claim 7, wherein the first probe is positioned in closer proximity to the aquatic system bed than the second probe.

9. The profile apparatus of claim 8, wherein the second probe is positioned in closer proximity to the aquatic system bed than the third probe.

10. The profile apparatus of claim 7, wherein the first probe, second probe and third probe are positioned such that they are within at least about 35 cm of the aquatic system bed.

11. The profile apparatus of claim 9, wherein the at least three dissolved oxygen probes are connected to the support such that they are aligned with respect to each other.

12. The profile apparatus of claim 1, wherein the water flow velocity measurement system comprises at least one velocimeter, and said at least one velocimeter measures water flow velocities in at least two directions.

13. The profile apparatus of claim 12, wherein the at least one velocimeter is an acoustic doppler velocimeter.

14. The profile apparatus of claim 1, further comprising a computer processor in communication with the dissolved oxygen measurement system and the water flow velocity measurement system, for receiving and processing data produced by the dissolved oxygen measurement system and the water flow velocity measurement system, to calculate the sediment oxygen demand of the aquatic system.

15. A profile apparatus for use in measuring sediment oxygen demand in an aquatic system, comprising:
(a) a support for positioning the profile apparatus in the aquatic system;
(b) a dissolved oxygen measurement system connected to the support for measuring a dissolved oxygen gradient;
(c) a first velocimeter for measuring the water flow velocity in a first direction, connected to the support and proximate to the dissolved oxygen measurement system;
(d) a second velocimeter for measuring the water flow velocity in a second direction orthogonal to the first direction and connected to the support and proximate to the dissolved oxygen measurement system; and
(e) a computer processor in communication with the dissolved oxygen measurement system, and the velocimeters, for receiving and processing data produced by the dissolved oxygen measurement system and the velocimeters to calculate the sediment oxygen demand of the aquatic system.

16. The profile apparatus of claim 15, further comprising a third velocimeter for measuring the water flow velocity in a third direction, said third velocimeter being orthogonal to the first and second directions, connected to the support and proximate to the dissolved oxygen measurement system.

17. A method for measuring sediment oxygen demand in an aquatic system, comprising the steps of:
(a) positioning the profile apparatus of claim 1 in the aquatic system;
(b) measuring a dissolved oxygen concentration for a pre-determined time period;
(c) measuring water flow velocities in at least two directions for said time period; and
(d) determining the sediment oxygen demand from the dissolved oxygen concentration, and the water flow velocities measured over said time period.

18. The method for measuring sediment oxygen demand in an aquatic system of claim 17, further comprising the steps of:
(a) measuring a dissolved oxygen gradient as a function of the dissolved oxygen concentration for said time period;
(b) measuring a friction velocity from the water flow velocities in at least two directions for said time period;
(c) determining a vertical eddy diffusivity coefficient as a function of friction velocity, an elevation above an aquatic system bed, and depth of the aquatic system, measured over said time period;
(d) determining a flux of the sediment oxygen demand as a function of the dissolved oxygen gradient and the vertical eddy diffusivity coefficient measured over said time period; and
(e) determining the sediment oxygen demand as a function of the flux of the sediment oxygen demand measured over said time period.

19. The method of claim 17, wherein said time period is at least about one minute to several days.

20. The method of claim 19, wherein said time period is at least about 5 to 15 minutes.

21. The method of claim 17, wherein the aquatic system comprises freshwater or saltwater.

22. The method of claim 17, wherein the aquatic system is a lotic system.

23. The method of claim 22, wherein the lotic system is selected from the group consisting of a river, stream, spring, creek and estuary.
